(19) United States
(12) Patent Application Publication
(10) Pub. No.: US 2003/0100790 A1
(43) Pub. Date: May 29, 2003

(54) Process for preparing N-formylamino carboxylic esters

(76) Inventors: Alexander Wartini, Heidelberg (DE); Eike Johannes Bergner, Schriesheim (DE); Klaus Ebel, Lampertheim (DE)

Correspondence Address: Herbert B. Keil, KEIL & WEINKAUF, 1350 Connecticut Ave., N.W., Washington, DC, 20036, US

(21) Appl. No.: 10/282,017

(22) Filed: Oct. 29, 2002

(30) Foreign Application Priority Data
Nov. 9, 2001 (DE) 10154716.1

(51) Int. Cl. C07C 69/74; C07C 231/02

(52) U.S. Cl. 560/41; 560/155

(57) ABSTRACT

The invention relates to a process for preparing N-formylamino carboxylic esters by reacting amino carboxylic acids with formic esters.

The invention relates to a process for preparing N-formylamino carboxylic esters.

N-Formylamino carboxylic esters are important precursors for preparing heterocycles such as oxazoles [Bull. Chem. Soc. Jpn. 1971, 44, 1407-1410], imidazoles [J. Med. Chem. 1969, 12(5), 804-806], or pyrazines [Chim. Ind. 1988, 70, 70-71].

The N-formylamino carboxylic esters of particular industrial importance are N-formylalanine butyl ester (FAB) and N-formylalanine ethyl ester because both compounds are precursors for preparing vitamin B₆ [Bull. Chem. Soc. Jpn. 1971, 44, 1407-1410]. FAB, for example, is prepared on the large scale in several 1000 tons.

The industrial synthesis starts by forming the hydrochloride of alanine with HCl, then reacting with butanol in the presence of hydrochloric acid to give the ester and subsequently in a further step formylating with formamide. The yield in the synthesis of FAB starting from alanine is about 90%. The associated disadvantages are corrosion problems due to gaseous hydrogen chloride and the formation of one equivalent of ammonium chloride as byproduct. There is also formation of industrially problematic byproducts such alkyl chlorides and dialkyl ethers.

Besides formulation with formamide, numerous other formulating reagents are described in the literature, such as, for example, formic acid [Bull. Chem. Soc. Jpn. 1972, 45, 1917-1918], the mixed anhydride of acetic acid and formic acid [Bull. Chem. Soc. Jpn. 1965, 38, 244-246], orthoformic esters [Synthesis 1994, 1023-1025] or cyanomethyl formate [Synthesis 1996, 1, 37-38].

However, all the cases described above start from the hydrochloride of the alanine ester, and a cost-effective, chlorine-free process is not then possible. In addition, these synthetic processes are complicated because they proceed over a plurality of stages.

A salt-free, one-stage synthesis of N-formylamino carboxylic esters starting from the amino acid is described in Bull. Chem. Soc. Jap. 1972, 45, 1917-1918. This entails heating the amino acid in the presence of formic acid and an alcohol to temperatures of from 120 to 180° C. in an autoclave. The yields achieved in this case are 35-71%, depending on the substitution pattern and alcohol used. These yields are unsatisfactory for large-scale use. The process has the additional disadvantage that large quantities of carboxylic esters are formed as waste product.

It is an object of the present invention to provide another, salt-free, one-stage process for preparing N-formylamino carboxylic esters which does not have the disadvantages of the prior art, can be used on a large-scale and provides the N-formylamino carboxylic esters in high yields, selectivities and with small quantities of byproducts.

We have found that this object is achieved by a process for preparing N-formylamino carboxylic esters by reacting amino carboxylic acids with formic esters.

Amino carboxylic acids mean in a manner known per se organic compounds which have a free amino function and a free carboxyl function. The process of the invention is not confined to particular amino carboxylic acids and can therefore be used for all amino carboxylic acids.

The amino carboxylic acids preferably used as amino carboxylic acids are selected from the group of compounds of the formula III and IV

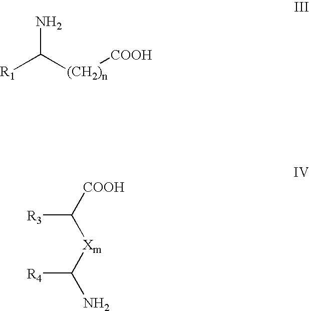

where

n is 0 to 12,

m is 0 to 4,

R₁ is hydrogen, a branched or unbranched, optionally substituted C₁-C₁₂-alkyl, C₂-C₁₂-alkenyl, C₂-C₁₂-alkynyl or C₁-C₆-alkylene-C₃-C₇-cycloalkyl radical, an optionally substituted C₃-C₇-cycloalkyl, aryl, arylalkyl, hetaryl or hetarylalkyl radical,

R₃ and R₄ form together via the radical Xₘ a 5- to 7-membered, optionally substituted, saturated, unsaturated or aromatic carbocycle or heterocycle which may contain up to three heteroatoms selected from the group of O, N or S,

X is (C—R₅) or (CH—R₅) and

R₅ are independently of one another, hydrogen, halogen, —NO₂, or —CN.

Formic esters mean in a manner known per se esters of formic acid with alcohols. The process of the invention is not confined to articular formic esters and can therefore be used for all formic esters. The formic ester is preferably employed as precursor in isolated form.

The formic esters preferably used as formic esters are of the formula V

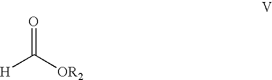

where

R₂ is a branched or unbranched, optionally substituted C₁-C₁₂-alkyl, C₂-C₁₂-alkenyl or C₂-C₁₂-alkynyl radical or an optionally substituted aryl or arylalkyl radical.

Optionally substituted radicals mean according to the invention the corresponding unsubstituted and substituted radicals. Suitable substituents for all substituted radicals of the present invention are, if not specified in detail, independently of one another up to 5 substituents selected, for example, from the following group:

—NO₂, —OH, —CN, halogen, a branched or unbranched, optionally substituted C₁-C₄-alkyl radical,

such as, for example, methyl, CF₃, C₂F₅ or CH₂F, a branched or unbranched, optionally substituted —CO—O—C₁-C₄-alkyl, C₃-C₇-cycloalkyl, C₁-C₄-alkoxy, C₁-C₄-alkylthio, —NH—CO—O—C₁-C₄-alkyl, —O—CH₂—COO—C₁-C₄-alkyl, —NH—CO—C₁-C₄-alkyl, —CO—NH—C₁-C₄-alkyl, —NH—SO₂—C₁-C₄-alkyl, —SO₂—NH—C₁-C₄-alkyl, —N(C₁-C₄-alkyl)₂, —NH—C₁-C₄-alkyl—, or —SO₂—C₁-C₄-alkyl radical, such as, for example, —SO₂—CF₃, an optionally substituted —NH—CO-aryl, —CO—NH-aryl, —NH—CO—O-aryl, —NH—CO—O-alkylenearyl, —NH—SO₂-aryl, —SO₂—NH-aryl, —CO—NH-benzyl, —NH—SO₂-benzyl or —SO₂—NH-benzyl radical.

In a preferred embodiment, n is 0 to 4, and with particular preference n is 0 or 1. When n is 0, the carboxyl carbon is directly adjacent to the α carbon as, for example, in natural amino acids.

In a further preferred embodiment, m is 0 to 2.

Branched or unbranched C₁-C₁₂-alkyl radicals for R₁ and R₂ are, independently of one another, for example methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, heptyl, octyl, nonyl, decyl, undecyl or dodecyl, preferably branched or unbranched C₁-C₄-alkyl radicals such as, for example methyl, ethyl, propyl, 1-methylethyl, butyl, 1-methylpropyl, 2-methylpropyl or 1,1-dimethylethyl, particularly preferably methyl.

A branched or unbranched C₂-C₁₂-alkenyl radical for R₁ and R₂ means, independently of one another, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-2-propenyl and the corresponding heptenyls, octenyls, nonenyls, decenyls, undecenyls and dodecenyls.

A branched or unbranched C₂-C₁₂-alkynyl radical for R₁ and R₂ means, independently of one another, for example ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl, and the corresponding heptynyls, octynyls, nonynyls, decynyls, undecynyls and dodecynyls.

A C₃-C₇-cycloalkyl radical for R₁ means, for example, cyclopropyl, cyclobutyl, cyclopentyl, cyclohexyl or cycloheptyl.

Branched or unbranched C₁-C₆-alkylene-C₃-C₇-cycloalkyl radicals are composed, for example, of branched or unbranched C₁-C₆-alkylene radicals and the aforementioned C₃-C₇-cycloalkyl radicals.

Preferred optionally substituted aryl radicals for R₁ and R₂ are, independently of one another, optionally substituted phenyl, 1-naphthyl or 2-naphthyl.

Preferred optionally substituted arylalkyl radicals for R₁ and R₂ are, independently of one another, optionally substituted benzyl or phenethyl.

Hetaryl radicals for R₁ mean, for example, radicals such as 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl.

Substituted hetaryl radicals for R₁ also mean fused derivatives of the aforementioned heteroaryl radicals, such as, for example, indazole, indole, benzothiophene, benzofuran, indoline, benzimidazole, benzthiazole, benzoxazole, quinoline, 2,3-dihydrobenzofuran, furo[2,3]pyridin, furo[3,2]pyridine or isoquinoline.

Hetarylalkyl radicals for R₁ mean radicals which are composed, for example, of C₁-C₆-alkylene radicals and of the hetaryl radicals described above, such as, for example, the radicals —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl, or —CH₂—CH₂-5-thiazolyl.

Preferred radicals for R₁ are hydrogen, optionally substituted C₁-C₁₂-alkyl, preferably C₁-C₆-alkyl, in particular C₁-C₄-alkyl, and optionally substituted aryl, preferably phenyl.

Particularly preferred radicals for R₁ are the side chains of natural amino acids, in particular hydrogen and methyl.

Preferred radicals for R₂ are optionally substituted C₁-C₁₂-alkyl, preferably C₁-C₆-alkyl, in particular C₁-C₄-alkyl, and optionally substituted aryl, preferably phenyl.

Particularly preferred radicals for R₂ are methyl, ethyl, n-propyl, isopropyl, n-butyl, isobutyl or tert-butyl, in particular methyl, ethyl and n-butyl.

The two radicals R₃ and R₄ form together via the radical Xₘ a 5- to 7-membered, optionally substituted, saturated, unsaturated or aromatic carbocycle or heterocycle which may contain up to three heteroatoms selected from the group of O, N or S.

X in this case is oxygen, sulfur, nitrogen, (C—R₅) or (CH—R₅), where the R₅ radicals are, independently of one another, hydrogen, halogen, —NO₂, or —CN.

In the case where X is oxygen, sulfur or nitrogen m is 1 or 2, preferably 1.

In the case where X is (C—R₅), X is part of an aromatic or unsaturated ring, with X being involved in a double bond.

In the case where X is (CH—R₅), X is part of a saturated or unsaturated ring, with X not being involved in a double bond.

For example, the radicals R₃ and R₄ can form together via the radical X (m=1) or the radicals X (m>1) an optionally substituted C₅-C₇-cycloalkyl radical such as, for example, cyclopentyl, cyclohexyl or cycloheptyl, an optionally substituted aryl radical such as, for example, phenyl, 1-naphthyl or 2-naphthyl, an optionally substituted C₅-C₇-heterocycloalkyl radical such as, for example, optionally substituted pyrrolidinyl, piperazinyl, morpholinyl, piperidinyl, tetrahydrofuranyl, tetrahydropyranyl, 1,4-dioxanyl, hexahydroazepinyl, oxepanyl, 1,2-oxathiolanyl or oxazolidinyl, an optionally substituted C₃-C₇-heterocycloalkenyl radical such as, for example, optionally substituted pyrrolinyls, oxazolinyls, azepinyl, oxepinyl, α-pyranyl, β-pyranyl, γ-pyranyl, dihydropyranyls, 2,5-dihydropyrrolyl or 4,5-dihydrooxazolyl, an optionally substituted hetaryl radical such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl or the fused derivatives thereof, such as, for example indazolyl, indolyl, benzothienyl, benzofuranyl, indolinyl, benzimidazolyl, benzothiazolyl, benzoxazolyl, quinolynyl or isoquinolynyl.

In a preferred embodiment, the two radicals R₃ and R₄ form together via the radical Xₘ a 5- to 7-membered aromatic carbocycle or heterocycle which may contain up to three heteroatoms selected from the group of O, N or S.

The particularly preferred amino carboxylic acids selected from the group of compounds of the formula III and IV are composed of the preferred radicals, described above, of the amino carboxylic acids. Particularly preferred amino carboxylic acids are the natural amino acids, in particular Ala, Arg, Asp, Cys, Phe, Gly, His, Ile, Leu, Met, Glu, Ser, Thr, Val, Trp and Tyr, particularly preferably alanine.

The amino carboxylic acids may be in enantiomer pure form, as racemic mixtures or in any ratios of stereoisomers.

The particularly preferred formic esters of the formula V are composed of the preferred radical R₂ described above. Very particular preferred formic esters are methyl formate, ethyl formate and n-butyl formate.

Accordingly, the invention preferably relates to a process for preparing N-formylamino carboxylic esters selected from the group of compounds of the formula I and II

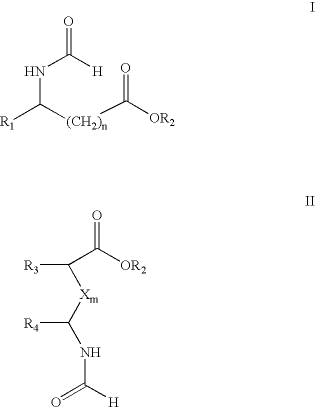

but reacting amino carboxylic acids selected from the group of compounds of the formula III and IV

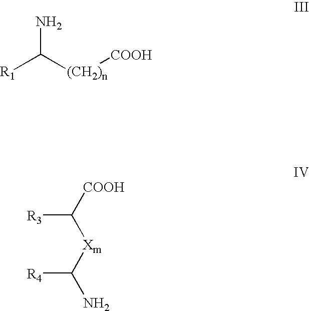

with formic esters of the formula V

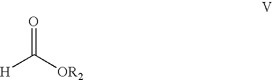

where the radicals have the meaning described above.

Preferred N-formylamino carboxylic esters selected from the group of compounds of the formula I and II results through use of the corresponding preferred amino carboxylic acids selected from the group of compounds of the formula III and IV and the corresponding preferred formic esters of the formula V as precursors in the process of the invention.

The temperature at which the process of the invention is carried out is not critical. Higher yields and selectivities advantageously result at temperatures above 110° C. The process is therefore preferably carried out at 110 to 200° C., particularly preferably at 140 to 180° C., very particularly preferably 155 to 165° C.

The pressure under which the process of the invention is carried out is not critical. In order to reach the advantageous temperatures of above 110° C. on use of precursors which boil below 110° C., it is advantageous to carry out the process under autogenous pressure or under a pressure above 1 bar. The pressure typically does not exceed 15 bar.

The process can be carried out particularly advantageously in an autoclave. In this case, the reaction mixture of amino carboxylic acid and formic ester is brought to the required temperature, preferably to the advantageous temperature described above, under autogenous pressure.

The molar ratio between amino carboxylic acid and formic ester is likewise not critical and is preferably 1:1 to 1:15, preferably 1:1 to 1:10. If the mole fraction of formic ester is greater than 0.5, a corresponding amount of formic ester is obtained as byproduct. The formic ester can be distilled out as low boiler and returned to the reaction.

In a further preferred embodiment, mixtures of formic ester of the formula V and the corresponding alcohol R₂—OH are used as formic esters of the formula V. The molar ratio of formic ester of the formula V to the appropriate alcohol R₂—OH is not critical and is typically 10:1 to 1:10, preferably 1:1 to 1:5.

The reaction time is not critical and is typically 4 to 24 hours, preferably 6 to 12 hours. Since the selectivity of the reaction is above 90% in every case, it is also possible carry out a process with partial conversion.

The N-formylamino carboxylic ester is normally separated from the precursor in a manner known per se by distillation, for example by fractional distillation.

The process of the invention results in high conversions, yields and selectivities compared with the prior art.

The following examples illustrate the invention without restricting the latter thereto.

### EXAMPLE 1

In an autoclave, 13.35 g (0.15 mol) of D,L-alanine were suspended in 90.1 g (1.5 mol) of methyl formate and stirred under nitrogen at 160° C. under autogenous pressure for 12 h. Unreacted alanine was filtered off and the solution was fractionally distilled. 18.5 g (0.141 mol) of N-formyl-D,L-alanine methyl ester were obtained. This corresponds to a yield of 94.2% with a selectivity of 97.5%.

### EXAMPLE 2

In an autoclave, 13.35 g (0.15 mol) of D,L-alanine were suspended in 111.1 g (1.5 mol) of ethyl formate and stirred under nitrogen at 160° C. under autogenous pressure for 12 h. Unreacted alanine was filtered off and the solution was fractionally distilled. 19.8 g (0.136 mol) of N-formyl-D,L-alanine ethyl ester were obtained. This corresponds to a yield of 90.9%.

### EXAMPLE 3

In an autoclave, 13.35 g (0.15 mol) of D,L-alanine were suspended in 122.4 g (1.2 mol) of butyl formate and stirred under nitrogen at 160° C. under autogenous pressure for 8 h. Unreacted alanine was filtered off and the solution was fractionally distilled. 23.5 g (0.136 mol) of N-formyl-D,L-alanine butyl ester (FAB) were obtained. This corresponds to a yield of 90.4%. EXAMPLE 4

In an autoclave, 13.35 g (0.15 mol) of D,L-alanine were suspended in a mixture of 85.7 g (0.84 mol) of butyl formate and 26.7 g (0.36 mol) of butanol and stirred under nitrogen at 160° C. under autogenous pressure for 8 h. Unreacted alanine was filtered off and the solution was fractionally distilled. 23.1 g (0.133 mol) of N-formyl-D,L-alanine butyl ester (FAB) were obtained. This corresponds to a yield of 88.8%.

A process for preparing N-formylamino carboxylic esters

The invention relates to a process for preparing N-formylamino carboxylic esters.

N-Formylamino carboxylic esters are important precursors for preparing heterocycles such as oxazoles [Bull. Chem. Soc. Jpn. 1971, 44, 1407-1410], imidazoles [J. Med. Chem. 1969, 12(5), 804-806], or pyrazines [Chim. Ind. 1988, 70, 70-71].

The N-formylamino carboxylic esters of particular industrial importance are N-formylalanine butyl ester (FAB) and N-formylalanine ethyl ester because both compounds are precursors for preparing vitamin B₆ [Bull. Chem. Soc. Jpn. 1971, 44, 1407-1410]. FAB, for example, is prepared on the large scale in several 1000 tons.

The industrial synthesis starts by forming the hydrochloride of alanine with HCl, then reacting with butanol in the presence of hydrochloric acid to give the ester and subsequently in a further step formylating with formamide. The yield in the synthesis of FAB starting from alanine is about 90%. The associated disadvantages are corrosion problems due to gaseous hydrogen chloride and the formation of one equivalent of ammonium chloride as byproduct. There is also formation of industrially problematic byproducts such alkyl chlorides and dialkyl ethers.

Besides formulation with formamide, numerous other formylating reagents are described in the literature, such as, for example, formic acid [Bull. Chem. Soc. Jpn. 1972, 45, 1917-1918], the mixed anhydride of acetic acid and formic acid [Bull. Chem. Soc. Jpn. 1965, 38, 244-246], orthoformic esters [Synthesis 1994, 1023-1025] or cyanomethyl formate [Synthesis 1996, 1, 37-38].

However, all the cases described above start from the hydrochloride of the alanine ester, and a cost-effective, chlorine-free process is not then possible. In addition, these synthetic processes are complicated because they proceed over a plurality of stages.

A salt-free, one-stage synthesis of N-formylamino carboxylic esters starting from the amino acid is described in Bull. Chem. Soc. Jap. 1972, 45, 1917-1918. This entails heating the amino acid in the presence of formic acid and an alcohol to temperatures of from 120 to 180° C. in an autoclave. The yields achieved in this case are 35-71%, depending on the substitution pattern and alcohol used. These yields are unsatisfactory for large-scale use. The process has the additional disadvantage that large quantities of carboxylic esters are formed as waste product.

It is an object of the present invention to provide another, salt-free, one-stage process for preparing N-formylamino carboxylic esters which does not have the disadvantages of the prior art, can be used on a large-scale and provides the N-formylamino carboxylic esters in high yields, selectivities and with small quantities of byproducts.

We have found that this object is achieved by a process for preparing N-formylamino carboxylic esters by reacting amino carboxylic acids with formic esters.

Amino carboxylic acids mean in a manner known per se organic compounds which have a free amino function and a free carboxyl function. The process of the invention is not confined to particular amino carboxylic acids and can therefore be used for all amino carboxylic acids.

The amino carboxylic acids preferably used as amino carboxylic acids are selected from the group of compounds of the formula III and IV

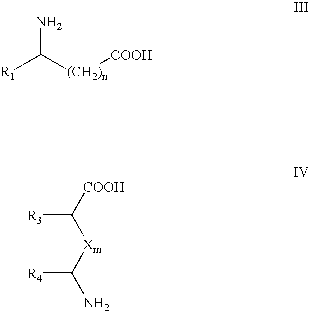

where

n is 0 to 12,

m is 0 to 4,

R₁ is hydrogen, a branched or unbranched, optionally substituted C₁-C₁₂-alkyl, C₂-C₁₂-alkenyl, C₂-C₁₂-alkynyl or C₁-C₆-alkylene-C₃-C₇-cycloalkyl radical, an optionally substituted C₃-C₇-cycloalkyl, aryl, arylalkyl, hetaryl or hetarylalkyl radical,

R₃ and R₄ form together via the radical Xₘ a 5- to 7-membered, optionally substituted, saturated, unsaturated or aromatic carbocycle or heterocycle which may contain up to three heteroatoms selected from the group of O, N or S,

X is (C—R₅) or (CH—R₅) and

R₅ are independently of one another, hydrogen, halogen, —NO₂, or —CN.

Formic esters mean in a manner known per se esters of formic acid with alcohols. The process of the invention is not confined to particular formic esters and can therefore be used for all formic esters. The formic ester is preferably employed as precursor in isolated form.

The formic esters preferably used as formic esters are of the formula V

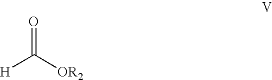

where

R₂ is a branched or unbranched, optionally substituted C₁-C₁₂-alkyl, C₂-C₁₂-alkenyl or C₂-C₁₂-alkynyl radical or an optionally substituted aryl or arylalkyl radical.

Optionally substituted radicals mean according to the invention the corresponding unsubstituted and substituted radicals. Suitable substituents for all substituted radicals of the present invention are, if not specified in detail, independently of one another up to 5 substituents selected, for example, from the following group:

—NO₂, —OH, —CN, halogen, a branched or unbranched, optionally substituted C₁-C₄-alkyl radical,

such as, for example, methyl, CF₃, C₂F₅ or CH₂F, a branched or unbranched, optionally substituted —CO—O—C₁-C₄-alkyl, C₃-C₇-cycloalkyl, C₁-C₄-alkoxy, C₁-C₄-alkylthio, —NH—CO—O—C₁-C₄-alkyl, —O—CH₂—COO—C₁-C₄-alkyl, —NH—CO—C₁-C₄-alkyl, —CO—NH—Cₗ-C₄-alkyl, —NH—SO₂—C₁-C₄-alkyl, —SO₂—NH—C₁-C₄-alkyl, —N(C₁-C₄-alkyl)₂, —NH—C₁-C₄-alkyl-, or —SO₂—C₁-C₄-alkyl radical, such as, for example, —SO₂—CF₃, an optionally substituted —NH—CO-aryl, —CO—NH-aryl, —NH—CO—O-aryl, —NH—CO—O-alkylenearyl, —NH—SO₂-aryl, —SO₂—NH-aryl, —CO—NH-benzyl, —NH—SO₂-benzyl or —SO₂—NH-benzyl radical.

In a preferred embodiment, n is 0 to 4, and with particular preference n is 0 or 1. When n is 0, the carboxyl carbon is directly adjacent to the a carbon as, for example, in natural amino acids.

In a further preferred embodiment, m is 0 to 2.

Branched or unbranched C₁-C₁₂-alkyl radicals for R₁ and R₂ are, independently of one another, for example methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, heptyl, octyl, nonyl, decyl, undecyl or dodecyl, preferably branched or unbranched C₁-C₄-alkyl radicals such as, for example methyl, ethyl, propyl, 1-methylethyl, butyl, 1-methylpropyl, 2-methylpropyl or 1,1-dimethylethyl, particularly preferably methyl.

A branched or unbranched C₂-C₁₂-alkenyl radical for R₁ and R₂ means, independently of one another, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-2-propenyl and the corresponding heptenyls, octenyls, nonenyls, decenyls, undecenyls and dodecenyls.

A branched or unbranched C₂-C₁₂-alkynyl radical for R₁ and R₂ means, independently of one another, for example ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl, and the corresponding heptynyls, octynyls, nonynyls, decynyls, undecynyls and dodecynyls.

A C₃-C₇-cycloalkyl radical for R₁ means, for example, cyclopropyl, cyclobutyl, cyclopentyl, cyclohexyl or cycloheptyl.

Branched or unbranched C₁-C₆-alkylene-C₃-C₇-cycloalkyl radicals are composed, for example, of branched or unbranched C₁-C₆-alkylene radicals and the aforementioned C₃-C₇-cycloalkyl radicals.

Preferred optionally substituted aryl radicals for R₁ and R₂ are, independently of one another, optionally substituted phenyl, 1-naphthyl or 2-naphthyl.

Preferred optionally substituted arylalkyl radicals for R₁ and R₂ are, independently of one another, optionally substituted benzyl or phenethyl.

Hetaryl radicals for R₁ mean, for example, radicals such as 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl.

Substituted hetaryl radicals for R₁ also mean fused derivatives of the aforementioned heteroaryl radicals, such as, for example, indazole, indole, benzothiophene, benzofuran, indoline, benzimidazole, benzthiazole, benzoxazole, quinoline, 2,3-dihydrobenzofuran, furo[2,3]pyridin, furo[3,2]pyridine or isoquinoline.

Hetarylalkyl radicals for R₁ mean radicals which are composed, for example, of C₁-C₆-alkylene radicals and of the hetaryl radicals described above, such as, for example, the radicals —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl, or —CH₂—CH₂-5-thiazolyl.

Preferred radicals for R₁ are hydrogen, optionally substituted C₁-C₁₂-alkyl, preferably C₁-C₆-alkyl, in particular C₁-C₄-alkyl, and optionally substituted aryl, preferably phenyl.

Particularly preferred radicals for R₁ are the side chains of natural amino acids, in particular hydrogen and methyl.

Preferred radicals for R₂ are optionally substituted C₁-C₁₂-alkyl, preferably C₁-C₆-alkyl, in particular C₁-C₄-alkyl, and optionally substituted aryl, preferably phenyl.

Particularly preferred radicals for R₂ are methyl, ethyl, n-propyl, isopropyl, n-butyl, isobutyl or tert-butyl, in particular methyl, ethyl and n-butyl.

The two radicals R₃ and R₄ form together via the radical Xₘ a 5- to 7-membered, optionally substituted, saturated, unsaturated or aromatic carbocycle or heterocycle which may contain up to three heteroatoms selected from the group of O, N or S.

X in this case is oxygen, sulfur, nitrogen, (C—R₅) or (CH—R₅), where the R₅ radicals are, independently of one another, hydrogen, halogen, —NO₂, or —CN.

In the case where X is oxygen, sulfur or nitrogen m is 1 or 2, preferably 1.

In the case where X is (C—R₅), X is part of an aromatic or unsaturated ring, with X being involved in a double bond.

In the case where X is (CH—R₅), X is part of a saturated or unsaturated ring, with X not being involved in a double bond.

For example, the radicals R₃ and R₄ can form together via the radical X (m=1) or the radicals X (m>1) an optionally substituted C₅-C₇-cycloalkyl radical such as, for example, cyclopentyl, cyclohexyl or cycloheptyl, an optionally substituted aryl radical such as, for example, phenyl, 1-naphthyl or 2-naphthyl, an optionally substituted C₁-C₇-heterocycloalkyl radical such as, for example, optionally substituted pyrrolidinyl, piperazinyl, morpholinyl, piperidinyl, tetrahydrofuranyl, tetrahydropyranyl, 1,4-dioxanyl, hexahydroazepinyl, oxepanyl, 1,2-oxathiolanyl or oxazolidinyl, an optionally substituted C₃-C₇-heterocycloalkenyl radical such as, for example, optionally substituted pyrrolinyls, oxazolinyls, azepinyl, oxepinyl, a-pyranyl, b-pyranyl, g-pyranyl, dihydropyranyls, 2,5-dihydropyrrolyl or 4,5-dihydrooxazolyl, an optionally substituted hetaryl radical such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl or the fused derivatives thereof, such as, for example indazolyl, indolyl, benzothienyl, benzofuranyl, indolinyl, benzimidazolyl, benzothiazolyl, benzoxazolyl, quinolynyl or isoquinolynyl.

In a preferred embodiment, the two radicals R₃ and R₄ form together via the radical Xₘ a 5- to 7-membered aromatic carbocycle or heterocycle which may contain up to three heteroatoms selected from the group of O, N or S.

The particularly preferred amino carboxylic acids selected from the group of compounds of the formula III and IV are composed of the preferred radicals, described above, of the amino carboxylic acids. Particularly preferred amino carboxylic acids are the natural amino acids, in particular Ala, Arg, Asp, Cys, Phe, Gly, His, Ile, Leu, Met, Glu, Ser, Thr, Val, Trp and Tyr, particularly preferably alanine.

The amino carboxylic acids may be in enantiomer pure form, as racemic mixtures or in any ratios of stereoisomers.

The particularly preferred formic esters of the formula V are composed of the preferred radical R₂ described above. Very particular preferred formic esters are methyl formate, ethyl formate and n-butyl formate.

Accordingly, the invention preferably relates to a process for preparing N-formylamino carboxylic esters selected from the group of compounds of the formula I and II

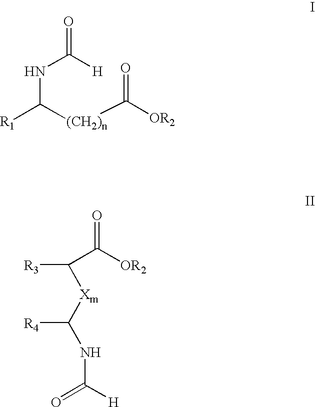

but reacting amino carboxylic acids selected from the group of compounds of the formula III and IV

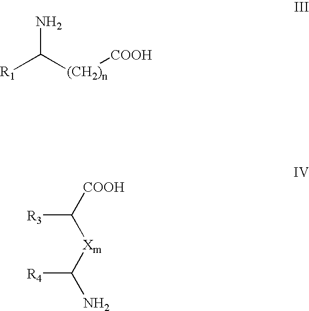

with formic esters of the formula V

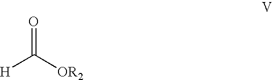

where the radicals have the meaning described above.

Preferred N-formylamino carboxylic esters selected from the group of compounds of the formula I and II results through use of the corresponding preferred amino carboxylic acids selected from the group of compounds of the formula III and IV and the corresponding preferred formic esters of the formula V as precursors in the process of the invention.

The temperature at which the process of the invention is carried out is not critical. Higher yields and selectivities advantageously result at temperatures above 110° C. The process is therefore preferably carried out at 110 to 200° C., particularly preferably at 140 to 180° C., very particularly preferably 155 to 165° C.

The pressure under which the process of the invention is carried out is not critical. In order to reach the advantageous temperatures of above 110° C. on use of precursors which boil below 110° C., it is advantageous to carry out the process under autogenous pressure or under a pressure above 1 bar. The pressure typically does not exceed 15 bar.

The process can be carried out particularly advantageously in an autoclave. In this case, the reaction mixture of amino carboxylic acid and formic ester is brought to the required temperature, preferably to the advantageous temperature described above, under autogenous pressure.

The molar ratio between amino carboxylic acid and formic ester is likewise not critical and is preferably 1:1 to 1:15, preferably 1:1 to 1:10. If the mole fraction of formic ester is greater than 0.5, a corresponding amount of formic ester is obtained as byproduct. The formic ester can be distilled out as low boiler and returned to the reaction.

In a further preferred embodiment, mixtures of formic ester of the formula V and the corresponding alcohol R₂—OH are used as formic esters of the formula V. The molar ratio of formic ester of the formula V to the appropriate alcohol R₂—OH is not critical and is typically 10:1 to 1:10, preferably 1:1 to 1:5.

The reaction time is not critical and is typically 4 to 24 hours, preferably 6 to 12 hours. Since the selectivity of the reaction is above 90% in every case, it is also possible carry out a process with partial conversion.

The N-formylamino carboxylic ester is normally separated from the precursor in a manner known per se by distillation, for example by fractional distillation.

The process of the invention results in high conversions, yields and selectivities compared with the prior art.

The following examples illustrate the invention without restricting the latter thereto.

### EXAMPLE 1

In an autoclave, 13.35 g (0.15 mol) of D,L-alanine were suspended in 90.1 g (1.5 mol) of methyl formate and stirred under nitrogen at 160° C. under autogenous pressure for 12 h. Unreacted alanine was filtered off and the solution was fractionally distilled. 18.5 g (0.141 mol) of N-formyl-D,L-alanine methyl ester were obtained. This corresponds to a yield of 94.2% with a selectivity of 97.5%.

### EXAMPLE 2

In an autoclave, 13.35 g (0.15 mol) of D,L-alanine were suspended in 111.1 g (1.5 mol) of ethyl formate and stirred under nitrogen at 160° C. under autogenous pressure for 12 h. Unreacted alanine was filtered off and the solution was fractionally distilled. 19.8 g (0.136 mol) of N-formyl-D,L-alanine ethyl ester were obtained. This corresponds to a yield of 90.9%.

### EXAMPLE 3

In an autoclave, 13.35 g (0.15 mol) of D,L-alanine were suspended in 122.4 g (1.2 mol) of butyl formate and stirred under nitrogen at 160° C. under autogenous pressure for 8 h. Unreacted alanine was filtered off and the solution was fractionally distilled. 23.5 g (0.136 mol) of N-formyl-D,L-alanine butyl ester (FAB) were obtained. This corresponds to a yield of 90.4%.

### EXAMPLE 4

In an autoclave, 13.35 9 (0.15 mol) of D,L-alanine were suspended in a mixture of 85.7 g (0.84 mol) of butyl formate and 26.7 g (0.36 mol) of butanol and stirred under nitrogen at 160° C. under autogenous pressure for 8 h. Unreacted alanine was filtered off and the solution was fractionally distilled. 23.1 g (0.133 mol) of N-formyl-D,L-alanine butyl ester (FAB) were obtained. This corresponds to a yield of 88.8%.

We claim:

1. A process for preparing N-formylamino carboxylic esters by reacting amino carboxylic acids with formic esters.

2. A process as claimed in claim 1, for preparing N-formylamino carboxylic esters selected from the group of compounds of formula I and II 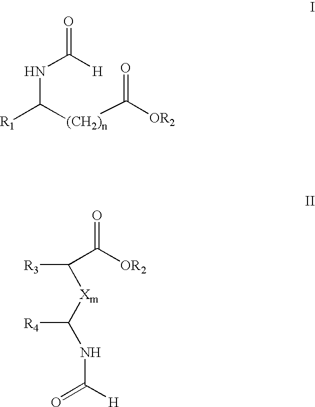
where
n is 0 to 12,
m is 0 to 4,
R₁ is hydrogen, a branched or unbranched, optionally substituted C₁-C₁₂-alkyl, C₂-C₁₂-alkenyl, C₂-C₁₂-alkynyl or C₁-C₆-alkylene-C₃-C₇-cycloalkyl radical, an optionally substituted C₃-C₇-cycloalkyl, aryl, arylalkyl, hetaryl or hetarylalkyl radical,
R₂ is a branched or unbranched, optionally substituted C₁-C₁₂-alkyl, C₂-C₁₂-alkenyl or C₂-C₁₂-alkynyl radical, an optionally substituted aryl or arylalkyl radical,
R₃ and R₄ form together via the radical Xₘ a 5- to 7-membered, optionally substituted, saturated, unsaturated or aromatic carbocycle or heterocycle which may contain up to three heteroatoms selected from the group of O, N or S,
X is O, S, N, (C—R₅) or (CH—R₅) and
R₅ are, independently of one another, hydrogen, halogen, —NO₂, or —CN
by reacting amino carboxylic acids selected from the group of compounds of the formula III and IV 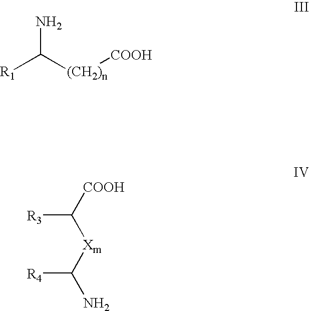
with formic esters of the formula V

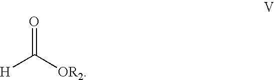

3. A process as claimed in claim 1 or 2, which is carried out at 110° C. to 200° C.

4. A process as claimed in any of claims 1 to 3, which, on use of precursors which boil below 110° C., is carried out under autogenous pressure or under a pressure above 1 bar.

5. A process as claimed in any of claims 1 to 4, wherein the molar ratio between amino carboxylic acid and formic ester is 1:1 to 1:10.

6. A process as claimed in any of claims 2 to 5, wherein mixtures of formic ester of the formula V and the appropriate alcohol R₂—OH are used as formic esters of the formula V.
